(19) United States
(12) Patent Application Publication
(10) Pub. No.: US 2005/0257293 A1
(43) Pub. Date: Nov. 17, 2005

(54) Biological containment system

(76) Inventor: Peter N. Mascia, Thousand Oaks, CA (US)

Correspondence Address: FISH & RICHARDSON P.C., PO BOX 1022, MINNEAPOLIS, MN, 55440-1022, US

(21) Appl. No.: 10/667,295

(22) Filed: Sep. 17, 2003

Related U.S. Application Data
(60) Provisional application No. 60/411,823, filed on Sep. 17, 2002.

(51) Int. Cl. A01H 1/00; C12N 15/82

(52) U.S. Cl. 800/287; 800/288

(57) ABSTRACT

The invention relates to materials and methods useful for controlling the unwanted spread of transgenic traits. The methods involve a male-sterile female containing a transgene for a desired trait and a transgene causing seed infertility. The methods also involve a male-fertile plant carrying a transcription activator that activates expression of both transgenes carried by the male-sterile female. Pollination of the male-sterile female by a male-fertile plant activates expression of both transgenes in the female. The resulting seeds express the gene product of the desired trait and are infertile.

This application claims priority to U.S. Provisional Application No. 60/411,823, filed Sep. 17, 2002, which is incorporated by reference in its entirety.

This application includes one compact disc, containing Sequence Tables and Reference Tables designated: sequences.311987.710-0004-55300-US-U-36440.01_{—}1; sequences.4565.710-0004-55300-US-U-36440.01_{—}1; sequences.3708.710-0004-55300-US-U-36440.01_{—}1; sequences.3769.710-0004-55300-US-U-36440.01_{—}1; sequences.3847.710-0004-55300-US-U-36440.01_{—}1; reference.4565.710-0004-55300-US-U-36440.01_{—}1; reference.3847.710-0004-55300-US-U-36440.01_{—}1; reference.3769.710-0004-55300-US-U-36440.01_{—}1; reference.3708.710-0004-55300-US-U-36440.01_{—}1; and reference.311987.710-0004-55300-US-U-36440.01_{—}1. The compact disc also contains an ortholog table designated ortholog.xls.

The compact disc also contains Consensus Sequences designated: 12514_gly_bra.txt; 12514.txt; 12653917.txt; 23771.txt; 3000_dico.txt; 3000.txt; 1610.txt; 519.txt; 8916.txt; 38419_mono.txt; 38419.txt; 38419_dico.txt; 32791.txt; 32348.txt; 5605.txt; 5605_gly₋bra.txt; and 519_gly.txt.

The compact disc also contains Matrix Tables designated 12514_gly_bra.matrix; 12514.matrix; 12653917.matrix; 23771.matrix; 3000_dico.matrix; 3000.matrix; 1610.matrix; 519.matrix; 8916.matrix; 38419_mono.matrix; 38419.matrix; 38419_dico.matrix; 32791.matrix; 32348.matrix; 5605.matrix; 5605_gly_bra.matrix; and 519_gly.matrix.

All of the above computer files are incorporated by reference in their entirety.

The invention relates to methods and materials for maintaining the integrity of the germplasm of transgenic and conventionally bred plants. In particular, the invention pertains to methods and materials that can be used to minimize the unwanted transmission of transgenic traits.

### BACKGROUND

Transgenic plants are now common in the agricultural industry. Such plants express novel transgenic traits such as insect resistance, stress tolerance, improved oil quality, improved meal quality and heterologous protein production. As more and more transgenic plants are developed and introduced into the environment, it is important to control the undesired spread of transgenic traits from transgenic plants to other traditional and transgenic cultivars, plant species and breeding lines.

While physical isolation and pollen trapping border rows have been employed to control transgenic plants under study conditions, these methods are cumbersome and are not practical for many cultivated transgenic plants. Effective ways to control the transmission and expression of transgenic traits without intervention would be useful for managing transgenic plants.

One recent genetic approach involves the production of transgenic plants that comprise recombinant traits of interest linked to repressible lethal genes. See, WO 00/37660. The lethal genes are blocked by the action of repressor molecules produced by repressor genes located at a different genetic locus. The lethal phenotype is expressed only if the repressible lethal gene construct and the repressor gene segregate after meiosis. This approach reportedly can be used to maintain genetic purity by blocking introgression of genes from plants that lack the repressor gene.

### SUMMARY

The present invention features methods and materials useful for controlling the transmission and expression of transgenic traits. The methods and materials of the invention facilitate the cultivation of transgenic plants without the undesired transmission of transgenic traits to other plants.

The invention features a method for making infertile seed. The method comprises permitting seed development to occur on a plurality of first plants that have been pollinated by a plurality of second plants. The first plants are male-sterile and comprise first and second nucleic acids. The first nucleic acid comprises a first transcription activator recognition site and a first promoter, operably linked to a sequence to be transcribed. The second nucleic acid comprises a second transcription activator recognition site and a second promoter, operably linked to a coding sequence causing seed infertility. The second plants are male-fertile and comprise at least one activator nucleic acid comprising at least one coding sequence for a transcription activator that is effective for binding to at least one of the above recognition sites. Each transcription activator coding sequence has a promoter operably linked thereto. The resulting seeds are infertile. The at least one activator nucleic acid can be a single nucleic acid encoding a single transcription activator that binds to both the first and second recognition sites. In some embodiments, the at least one activator nucleic acid is two nucleic acids, each encoding different transcription activators, one of which can bind the first recognition site and the other of which can bind the second recognition site. Alternatively, the at least one activator nucleic acid can be a single nucleic acid encoding a first transcription activator that can bind the first recognition site and encoding a second transcription activator that can bind the second recognition site. The promoter for the transcription activator can be seed-specific, or can be chemically inducible. The plants can be dicotyledonous plants, or monocotyledonous plants. The method can further comprise the step of harvesting the seeds. The plurality of first plants can be cytoplasmically male-sterile, or genetically male-sterile.

In some embodiments, the sequence to be transcribed encodes a preselected polypeptide, and the seeds can have a statistically significant increase in the amount of the preselected polypeptide relative to seeds that do not contain or express the first nucleic acid. The preselected polypeptide can be an antibody, or an industrial enzyme.

The sequence causing seed infertility can encode a seed infertility polypeptide, such as a loss-of-function mutant FIE polypeptide, a LEC2 polypeptide, an ANT polypeptide, or a LEC1 polypeptide.

The invention also features a method for making a polypeptide, which comprises obtaining seed produced by pollination of a male-sterile plant. Such seed comprises a first nucleic acid comprising a first recognition site for a transcription activator and a first promoter, operably linked to a sequence to be transcribed. Such seed also comprises a second nucleic acid comprising a second recognition site for a transcription activator and a second promoter, operably linked to a sequence causing seed infertility. Such seed also comprises at least one activator nucleic acid comprising at least one coding sequence for a transcription activator that binds to at least one of said recognition sites, each of the at least one transcription activators having a promoter operably linked thereto. The seeds are infertile and have a statistically significant increase in the amount of an endogenous polypeptide relative to seeds that do not contain or express said first nucleic acid. The endogenous polypeptide can be extracted from the seed.

A method for making a polypeptide can comprise permitting a plurality of first, male-sterile, plants to be pollinated by a plurality of second plants. The first plants comprise a first nucleic acid comprising a first transcription activator recognition site and a first promoter, operably linked to a coding sequence encoding a preselected polypeptide; and a second nucleic acid comprising a second transcription activator recognition site and a second promoter, operably linked to a sequence causing seed infertility. The second plants comprise at least one activator nucleic acid encoding at least one transcription activator that binds to at least one of the recognition sites. Each of the at least one transcription activators has a promoter operably linked thereto. The method also comprises harvesting seeds from the plurality of first plants. The resulting said seeds are infertile and have a statistically significant increase in the amount of preselected polypeptide relative to seeds that do not contain or express the first nucleic acid. The method can also comprise extracting the preselected polypeptide from the seeds. The plurality of first plants and said plurality of second plants can be randomly interplanted.

The invention also features an article of manufacture, which comprises a container, a first type of seeds within the container, and a second type of seeds within the container. The first type of seeds comprise at least one first nucleic acid comprising a first transcription activator recognition site and a first promoter, operably linked to a sequence to be transcribed, and a second transcription activator recognition site and a second promoter, operably linked to a sequence causing seed infertility. Plants grown from the first type of seeds are male-sterile. The second type of seeds comprise at least one activator nucleic acid, which encodes one or more transcription activators that are effective for binding to a corresponding one or more of the recognition sites, each transcription activator coding sequence has a promoter operably linked thereto. Plants grown from the second type of seeds are male-fertile. The sequence to be transcribed can encode a preselected polypeptide. The ratio of the first type of seeds to the second type of seeds can be about 70:30 or greater. The first and second types of seeds can be monocotyledonous seeds or dicotyledonous seeds. The invention also features a plant grown from one of the above types of seeds.

The inventions also features a nucleic acid construct comprising a first transcription activator recognition site and a first promoter. The first recognition site and first promoter are operably linked to a sequence to be transcribed. The nucleic acid construct also comprises a second transcription activator recognition site and a second promoter, each of which are operably linked to a second coding sequence encoding a seed infertility factor. The sequence causing seed infertility can be transcribed into a FIE antagonist, e.g., a FIE antisense RNA, or a ribozyme, or a chimeric polypeptide comprising a polypeptide segment exhibiting histone acetyltransferase activity fused to a polypeptide segment exhibiting activity of a subunit of a chromatin-associated protein complex having histone deacetylase activity. The sequence to be transcribed in the nucleic acid construct can encode a preselected polypeptide, e.g., an antibody, a polypeptide that has immunogenic activity in a mammal, or an industrial enzyme such as glucose-6-phosphate dehydrogenase or alpha-amylase. The sequence causing seed infertility can encode a LEC2 polypeptide, an ANT polypeptide or a LEC1 polypeptide.

The invention also features a method for making infertile seed. A plurality of male-sterile first plants are provided for the method, each such plant comprising a first nucleic acid and a second nucleic acid. The first nucleic acid comprises a first transcription activator recognition site and a first promoter. The first recognition site and the first promoter are operably linked to a sequence to be transcribed. The second nucleic acid comprises a second transcription activator recognition site and a second promoter. The second recognition site and the second promoter are operably linked to a sequence that results in seed infertility. A plurality of male-fertile second plants are provided for the method, each such plant comprising at least one activator nucleic acid. The activator nucleic acid comprises at least one coding sequence for a transcription activator that binds to at least one of the recognition sites, and each at least one transcription activator coding sequence has a promoter operably linked to it. Seed development is permitted to occur on the first plants after pollination by pollen from the second plants. The seeds are infertile such that the seeds produce no seedlings or seedlings that are not fertile.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description.

### BRIEF DESCRIPTION OF TABLES

Tables—Reference Tables

Sequences useful in the instant invention are described in the Sequence Tables and Reference Tables (sometimes referred to as REF Table). Sequence Tables are found in computer files named:
sequences.311987.710-0004-55300-US-U-36440.01_{—}1;
sequences.4565.710-0004-55300-US-U-36440.01_{—}1;
sequences.3708.710-0004-55300-US-U-36440.01_{—}1;
sequences.3769.710-0004-55300-US-U-36440.01_{—}1; and
sequences.3847.710-0004-55300-US-U-36440.01_{—}1.

Reference Tables are found in computer files designated:
reference.4565.710-0004-55300-US-U-36440.01_{—}1;
reference.3847.710-0004-55300-US-U-36440.01_{—}1;
reference.3769.710-0004-55300-US-U-36440.01_{—}1;
reference.3708.710-0004-55300-US-U-36440.01_{—}1; and
reference.311987.710-0004-55300-US-U-36440.01_{—}1.

A Reference Table refers to a number of “Maximum Length Sequences” or “MLS.” Each MLS corresponds to the longest cDNA and is described in the Av subsection of the Reference Table. The Reference Table includes the following information relating to each MLS:

| | |
|---|---|
| I. | cDNA Sequence |

| | |
|---|---|
| A. | 5′ UTR |
| B. | Coding Sequence |
| C. | 3′ UTR |

| | |
|---|---|
| II. | Genomic Sequence |

| | |
|---|---|
| A. | Exons |
| B. | Introns |
| C. | Promoters |

| | |
|---|---|
| III. | Link of cDNA Sequences to Clone IDs |
| IV. | Multiple Transcription Start Sites |
| V. | Polypeptide Sequences |

| | |
|---|---|
| A. | Signal Peptide |
| B. | Domains |
| C. | Related Polypeptides |

| | |
|---|---|
| VI. | Related Polynucleotide Sequences |

I. cDNA Sequence

The Reference Table indicates which sequence in the Sequence Table represents the sequence of each MLS. The MLS sequence can comprise 5′ and 3′ UTR as well as coding sequences. In addition, specific cDNA clone numbers also are included in the Reference Table when the MLS sequence relates to a specific cDNA clone.

A. 5′ UTR

The location of the 5′ UTR can be determined by comparing the most 5′ MLS sequence with the corresponding genomic sequence as indicated in the Reference Table. The sequence that matches, beginning at any of the transcriptional start sites and ending at the last nucleotide before any of the translational start sites corresponds to the 5′ UTR.

B. Coding Region

The coding region is the sequence in any open reading frame found in the MLS. Coding regions of interest are indicated in the PolyP SEQ subsection of the Reference Table.

C. 3′ UTR

The location of the 3′ UTR can be determined by comparing the most 3′ MLS sequence with the corresponding genomic sequence as indicated in the Reference Table. The sequence that matches, beginning at the translational stop site and ending at the last nucleotide of the MLS corresponds to the 3′ UTR.

II. Genomic Sequence

Further, the Reference Table indicates the specific “gi” number of the genomic sequence if the sequence resides in a public databank. For each genomic sequence, Reference tables indicate which regions are included in the MLS. These regions can include the 5′ and 3′ UTRs as well as the coding sequence of the MLS. See, for example, the scheme below:

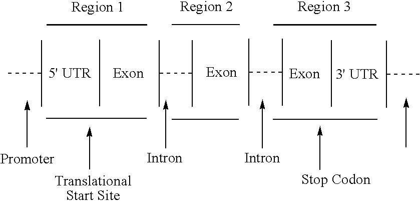

The Reference Table reports the first and last base of each region that are included in an MLS sequence. An example is shown below:
gi No. 47000:
37102 . . . 37497
37593 . . . 37925

The numbers indicate that the MLS contains the following sequences from two regions of gi No. 47000; a first region including bases 37102-37497, and a second region including bases 37593-37925.

A. Exon Sequences

The location of the exons can be determined by comparing the sequence of the regions from the genomic sequences with the corresponding MLS sequence as indicated by the Reference Table.

i. Initial Exon

To determine the location of the initial exon, information from the
(1) polypeptide sequence section;
(2) cDNA polynucleotide section; and
(3) the genomic sequence section

of the Reference Table is used. First, the polypeptide section will indicate where the translational start site is located in the MLS sequence. The MLS sequence can be matched to the genomic sequence that corresponds to the MLS. Based on the match between the MLS and corresponding genomic sequences, the location of the translational start site can be determined in one of the regions of the genomic sequence. The location of this translational start site is the start of the first exon.

Generally, the last base of the exon of the corresponding genomic region, in which the translational start site was located, will represent the end of the initial exon. In some cases, the initial exon will end with a stop codon, when the initial exon is the only exon.

In the case when sequences representing the MLS are in the positive strand of the corresponding genomic sequence, the last base will be a larger number than the first base. When the sequences representing the MLS are in the negative strand of the corresponding genomic sequence, then the last base will be a smaller number than the first base.

ii. Internal Exons

Except for the regions that comprise the 5′ and 3′ UTRs, initial exon, and terminal exon, the remaining genomic regions that match the MLS sequence are the internal exons. Specifically, the bases defining the boundaries of the remaining regions also define the intron/exon junctions of the internal exons.

iii. Terminal Exon

As with the initial exon, the location of the terminal exon is determined with information from the
(1) polypeptide sequence section;
(2) cDNA polynucleotide section; and
(3) the genomic sequence section

of the Reference Table. The polypeptide section will indicate where the stop codon is located in the MLS sequence. The MLS sequence can be matched to the corresponding genomic sequence. Based on the match between MLS and corresponding genomic sequences, the location of the stop codon can be determined in one of the regions of the genomic sequence. The location of this stop codon is the end of the terminal exon. Generally, the first base of the exon of the corresponding genomic region that matches the cDNA sequence, in which the stop codon was located, will represent the beginning of the terminal exon. In some cases, the translational start site will represent the start of the terminal exon, which will be the only exon.

In the case when the MLS sequences are in the positive strand of the corresponding genomic sequence, the last base will be a larger number than the first base. When the MLS sequences are in the negative strand of the corresponding genomic sequence, then the last base will be a smaller number than the first base.

B. Intron Sequences

In addition, the introns corresponding to the MLS are defined by identifying the genomic sequence located between the regions where the genomic sequence comprises exons. Thus, introns are defined as starting one base downstream of a genomic region comprising an exon, and end one base upstream from a genomic region comprising an exon.

C. Promoter Sequences

As indicated below, promoter sequences corresponding to the MLS are defined as sequences upstream of the first exon; more usually, as sequences upstream of the first of multiple transcription start sites; even more usually as sequences about 2,000 nucleotides upstream of the first of multiple transcription start sites.

III. Link of cDNA Sequences to Clone IDs

As noted above, the Reference Table identifies the cDNA clone(s) that relate to each MLS. The MLS sequence can be longer than the sequences included in the cDNA clones. In such a case, the Reference Table indicates the region of the MLS that is included in the clone. If either the 5′ or 3′ termini of the cDNA clone sequence is the same as the MLS sequence, no mention will be made.

IV. Multiple Transcription Start Sites

Initiation of transcription can occur at a number of sites of the gene. The Reference Table indicates the possible multiple transcription sites for each gene. In the Reference Table, the location of the transcription start sites can be either a positive or negative number.

The positions indicated by positive numbers refer to the transcription start sites as located in the MLS sequence. The negative numbers indicate the transcription start site within the genomic sequence that corresponds to the MLS.

To determine the location of the transcription start sites with the negative numbers, the MLS sequence is aligned with the corresponding genomic sequence. In the instances when a public genomic sequence is referenced, the relevant corresponding genomic sequence can be found by direct reference to the nucleotide sequence indicated by the “gi” number shown in the public genomic DNA section of the Reference Table. When the position is a negative number, the transcription start site is located in the corresponding genomic sequence upstream of the base that matches the beginning of the MLS sequence in the alignment. The negative number is relative to the first base of the MLS sequence which matches the genomic sequence corresponding to the relevant “gi” number.

In the instances when no public genomic DNA is referenced, the relevant nucleotide sequence for alignment is the nucleotide sequence associated with the amino acid sequence designated by “gi” number of the later PolyP SEQ subsection.

V. Polypeptide Sequences

The PolyP SEQ subsection lists SEQ ID NOS. and Ceres SEQ ID NO for polypeptide sequences corresponding to the coding sequence of the MLS sequence and the location of the translational start site with the coding sequence of the MLS sequence.

The MLS sequence can have multiple translational start sites and can be capable of producing more than one polypeptide sequence.

Subsection (Dp) provides (where present) information concerning amino acid sequences that are found to be related and have some percentage of sequence identity to the polypeptide sequences of the Reference and Sequence Tables. These related sequences are identified by a “gi” number.

Tables—Protein Group Matrix Tables

In addition to each consensus sequence of the invention, Applicants have generated scoring matrices in Matrix Tables to provide further description of a consensus sequence. The Matrix Tables can be found in computer files: 12514_gly_bra.matrix; 12514.matrix; 12653917.matrix; 23771.matrix; 3000_dico.matrix; 3000.matrix; 1610.matrix; 519.matrix; 8916.matrix; 38419_mono.matrix; 38419.matrix; 38419_dico.matrix; 32791.matrix; 32348.matrix; 5605.matrix; 5605_gly_bra.matrix; and 519_gly.matrix. The first row of each matrix indicates the residue position in the consensus sequence. The matrix reports the number of occurrences of all the amino acids that were found in the group members for every residue position of the signature sequence. The matrix also indicates for each residue position, how many different organisms were found to have a polypeptide in the group that included a residue at the relevant position. The last line of the matrix indicates all the amino acids that were found at each position of the consensus. The consensus sequence for each of the above Matrix Tables are in the corresponding Consensus Sequence Table. The Consensus Sequence Tables can be found in computer files: 12514_gly_bra.txt; 12514.txt; 12653917.txt; 23771.txt; 3000_dico.txt; 3000.txt; 1610.txt; 519.txt; 8916.txt; 38419_mono.txt; 38419.txt; 38419_dico.txt; 32791.txt; 32348.txt; 5605.txt; 5605_gly_bra.txt; and 519 gly.txt.

### DETAILED DESCRIPTION

The invention provides novel genetic methods and tools for effectively controlling the transmission of recombinant DNA-based traits from transgenic plants to other cultivars. The invention is based, in part, on the discovery that coordinate expression of certain nucleic acid constructs can control outcrossing and expression of transgenic traits. The method results in the production of infertile seed that carry a gene product for a desired trait. The infertility of the seed prevents unwanted spread of the desired transgenic trait.

Methods for Making Infertile Seed

In one aspect, the invention features a method for making infertile seed. The method comprises permitting seed development to occur on a plurality of first plants that have been pollinated by a plurality of second plants. The first plants are male-sterile and comprise first and second nucleic acids. The first nucleic acid comprises a first transcription activator recognition site and a first promoter, that are operably linked to a sequence to be transcribed into a desired gene product. The second nucleic acid comprises a second transcription activator recognition site and a second promoter, that are operably linked to a coding sequence causing seed infertility.

The second plants are male-fertile and comprise at least one activator nucleic acid encoding at least one transcription activator and a promoter operably linked thereto. In some embodiments, the transcription activator is effective for binding to both the first and second recognition sites. Upon pollination of the first, male-sterile plants by pollen from the second, male-fertile plants, seed development ensues. The activator nucleic acid carried by the pollen is expressed prior to or during seed development, and the resulting transcription activator activates transcription of the first and the second nucleic acids in developing seeds on the male-sterile female plants. Transcription of the first nucleic acid results in the production of a desired gene product in the resulting seeds, while transcription of the second nucleic acid causes seed infertility. The desired gene product present in the seeds is contained because all, or substantially all, of the seeds are infertile. Thus, unwanted spread of the transgene responsible for the desired trait to the environment, and the desirable trait is effectively contained.

All, or substantially all, of the resulting seeds have a statistically significant increase in the amount of the desired gene product relative to seeds that do not contain or express the first nucleic acid. Seeds made by the method contain the first, the second and the third nucleic acid.

In some embodiments, a single activator nucleic acid encodes two different transcription activators, one of which binds to the first recognition site and the other of which binds to the second recognition site. Alternatively, two different transcription activators can be encoded by separate nucleic acids. In either case, each of the transcription activators can have a different expression pattern, e.g., the transcription activator for the first recognition site can be operably linked to a constitutive promoter and the transcription activator for the second recognition site can be operably linked to a seed-specific promoter. In other embodiments, both transcription activators are operably linked to different, seed-specific promoters.

Desired gene products. Typically, the desired gene product of a sequence to be transcribed is a preselected polypeptide. A preselected polypeptide can be any polypeptide (i.e., 5 or more amino acids joined by a peptide bond). Plants have been used to produce a variety of preselected industrial and pharmaceutical polypeptides, including high value chemicals, modified and specialty oils, enzymes, renewable non-foods such as fuels and plastics, vaccines and antibodies. See e.g., Owen, M. and Pen, J. (eds.), 1996. Transgenic Plants: A Production System for Industrial and Pharmaceutical Proteins. John Wiley & Son Ltd.; Austin, S. et al., 1994. Annals NY Acad. Sci. 721:234-242; Austin, S. et al., 1995. Euphytica 85: 381-393; Ziegelhoffer, T. et al., 1998. Molecular Breeding. U.S. Pat. No. 5,824,779 discloses phytase-protein-pigmenting concentrate derived from green plant juice. U.S. Pat. No. 5,900,525 discloses animal feed compositions containing phytase derived from transgenic alfalfa. U.S. Pat. No. 6,136,320 discloses vaccines produced in transgenic plants. U.S. Pat. No. 6,255,562 discloses insulin. U.S. Pat. No. 5,958,745 discloses the formation of copolymers of 3-hydroxy butyrate and 3-hydroxy valerate. U.S. Pat. No. 5,824,798 discloses starch synthases. U.S. Pat. No. 6,303,341 discloses immunoglobulin receptors. U.S. Pat. No. 6,417,429 discloses immunoglobulin heavy- and light-chain polypeptides. U.S. Pat. No. 6,087,558 discloses the production of proteases in plants. U.S. Pat. No. 6,271,016 discloses an anthranilate synthase gene for tryptophan overproduction in plants.

A preselected polypeptide can be an antibody or antibody fragment. An antibody or antibody fragment includes a humanized or chimeric antibody, a single chain Fv antibody fragment, an Fab fragment, and an F(ab)₂ fragment. A chimeric antibody is a molecule in which different portions are derived from different animal species, such as those having a variable region derived from a mouse monoclonal antibody and a human immunoglobulin constant region. Antibody fragments that have a specific binding affinity can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Plant glycans are often non-immunogenic in animals or humans. However, if desired, glycosylation sites can be identified in a preselected polypeptide, and relevant glycosyl transferases can be expressed in parallel with expression of the preselected polypeptide. Alternatively, it may be desirable to prevent glycosylation of a preselected polypeptide, by engineering N-acetylglucosaminyltransferase knock-out plants. If a preselected polypeptide is an antibody or antibody fragment, Asn-X-Ser/Thr sites in the antibody can be deleted.

In some embodiments, the gene product of a sequence to be transcribed is one of the preselected polypeptides in the Table below.

TABLE 1

| | | |
|---|---|---|
| Bromelain | Humatrope ® | Proleukin ® |
| Chymopapain | Humulin ® (insulin) | Protropin ® |
| Papain ® | Infergen ® | Recombivax-HB ® |
| Activase ® | Interferon-gamma-1a | Recormon ® |
| Albutein ® | Interlekin-2 | Remicade ® (s-TNF-r) |
| Angiotensis II | Intron ® | ReoPro ® |
| Asparaginase | Leukine ® (GM-CSF) | Retavase ® (TPA) |
| Avonex ® | Nartogastrim ® | Roferon-A ® |
| Betaseron ® | Neumega ® | Pegaspargas |
| BioTropin ® | Neupogen ® | Prandin ® |
| Cerezyme ® | Norditropin ® | Procrit ® |
| Enbrel ® (s-TNF-r) | Novolin ® (insulin) | Filgastrim ® |
| Engerix-B ® | Nutropin ® | Genotropin ® |
| Epogen ® | Oncaspar ® | Geref ® |
| Sargramostrim | Tripedia ® | Trichosanthin |
| TriHIBit ® | Venoglobin-S ® (HIG) | |

In some embodiments, a sequence to be transcribed results in a desired gene product that is an RNA. Such an RNA, made from a sequence to be transcribed, can be useful for inhibiting expression of an endogenous gene. Suitable DNAs from which such an RNA can be made include an antisense construct and a co-suppression construct. Thus, for example, a sequence to be transcribed can be similar or identical to the sense coding sequence of an endogenous polypeptide, but is transcribed into a mRNA that is unpolyadenylated, lacks a 5′ cap structure, or contains an unsplicable intron. Alternatively, a sequence to be transcribed can incorporate a sequence encoding a ribozyme. In another alternative, a sequence to be transcribed can include a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of an endogenous polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of an endogenous polypeptide, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050. See, e.g., WO 98/53083; WO 99/32619; WO 98/36083; and WO 99/53050. See also, U.S. Pat. No. 5,034,323. Useful RNA gene products are described in, e.g., U.S. Pat. No. 6,326,527.

It will be recognized that more than one sequence to be transcribed can be present in some embodiments. For example, coding sequences for two preselected polypeptides may be present on the same or different nucleic acids, and encode polypeptides useful for manipulating a biosynthetic pathway. Alternatively, two coding sequences may be present and encode polypeptides found in a single protein, e.g., a heavy-chain immunoglobulin polypeptide and a light-chain immunoglobulin polypeptide, respectively.

Sequence causing seed infertility. A nucleic acid that results in seed infertility can encode a polypeptide, e.g., a polypeptide involved in seed development, or can form a transcription product. Overexpression or timely expression of such a nucleic acid results in the production of infertile seeds, i.e., seeds that are incapable of producing offspring. In some embodiments, infertile seeds do not germinate. In other embodiments, infertile seeds germinate and form seedlings that do not mature, e.g., seedlings that die before reaching maturity. In yet other embodiments, infertile seeds germinate and form mature plants that are incapable of forming seeds, e.g., that produce no floral structures or abnormal floral structures, or that cannot form gametes.

The product of a nucleic acid that results in seed infertility, i.e., a seed infertility factor, can be an agonist of a polypeptide involved in seed development. Such agonists can be polypeptides (e.g., dominant loss-of-function mutants), and also can be nucleic acids (e.g., antisense nucleic acids, ribozymes, or double-stranded RNA). Those skilled in the art can construct dominant loss of function mutants or nucleic acids using routine methods. Disruption of the function of polypeptides involved in seed development can result in the production of infertile seeds. Polypeptides involved in seed development can be identified, for example, by review of the scientific literature for reports of such polypeptides, by identifying orthologs of polypeptides reportedly involved in seed development, and by genetic screening. Certain nucleic acids suitable for use in conferring seed infertility are described in the Sequence Tables and Reference Tables. See also Table 2 below, which lists clone IDs for some such nucleic acids. Orthologs of these nucleic acids are found in the computer file ortholog.xls.

TABLE 2
Clone ID

clone 32791
clone 332
clone 519
clone 23771
clone 3000
clone 32791
clone 32348
clone 12514
clone 1610
clone 248859
clone 3858
clone 8916
clone 38419
clone 5605
cDNA 1821568

An exemplary polypeptide involved in seed development is the FIE polypeptide, which suppresses endosperm development until fertilization occurs. See, U.S. Pat/ No. 6,229,064. Seeds that inherit a mutant Fie allele are reported to abort, even if the paternal allele is normal. See, Yadegari, R. et al., Plant Cell 12:2367-81 (2000); U.S. Pat. No. 6,093,874. Other polypeptides for which suppression of expression can cause seed infertility include the products of the DMT and MEA genes. Another exemplary polypeptide involved in seed development is AP2, which is reportedly required for normal seed development. See, U.S. Pat. No. 6,093,874. Two other exemplary polypeptides involved in seed development are INO and ANT, which reportedly are required for ovule integument development. Mutations in INO and ANT reportedly can affect ovule development, resulting in incomplete megasporogenesis. See, WO 00/40694. Thus, transgenes encoding dominant negative suppression polypeptides, or transgenes producing antisense, ribozyme or double stranded RNA gene products can cause seed infertility.

Another exemplary polypeptide involved in seed development is the polypeptide encoded by the LEC2 gene. LEC2 and LEC2-orthologous polypeptides are transcription factors that typically possess a DNA binding domain termed the B3 domain. See, e.g., amino acid residues 165 to 277 in SEQ ID NO:2 of U.S. Pat. No. 6,492,577. A B3 domain can be found in other transcription factors including VIVIPAROUS1, AUXIN RESPONSE FACTOR 1, FUSCA3 and ABI3. Mutations in the LEC2 polypeptide are thought to cause defects in the late seed maturation phase of embryo development.

Another polypeptide involved in seed development is a HAP3-type CCAAT-box binding factor (CBF) subunit. A CBF complex is a heteromeric complex that binds a promoter element having a CCAAT nucleotide sequence motif, often found in the 5′ region of eukaryotic genes. CBF complexes bind the CCAAT motif in a wide variety of organisms. CBF complexes include at least two subunits that are involved in binding DNA, as well as one or more subunits that have transcription activation activity. The HAP3-type CBF subunits listed in Table 3 are homologous to the Arabidopsis thaliana HAP3 subunit having GI accession number 3282674. This particular HAP3 type CBF subunit is encoded by the Arabidopsis LEAFY COTYLEDON1 (LEC1) gene, which is reportedly required for the specification of cotyledon identity and the completion of embryo maturation. See, e.g., U.S. Pat. Nos. 6,320,102 and 6,235,974. The LEC1 gene reportedly functions at an early developmental stage to maintain embryonic cell fate. LEC1 RNA accumulates during seed development in embryo cell types and in endosperm tissue. Ectopic postembryonic expression of the LEC1 gene in vegetative cells induces the expression of embryo-specific genes and initiates formation of embryo-like structures. Thus LEC1 appears to be an important regulator of embryo development that activates the transcription of genes required for both embryo morphogenesis and cellular differentiation. Also indicative of LEC1's role in seed maturation are the observations that lec1 mutant seed have altered morphology. For example, during seed development the shoot meristem is activated prematurely. Moreover, the embryo does not synthesize seed storage proteins. Finally lec1 seed are desiccation intolerant and die during late embryogenesis. LEC1 CBF subunits can be distinguished from other HAP3-type subunits on the basis of at least one diagnostic conserved sequence. See e.g., WO 99/67405 and WO/00/28058.

TABLE 3
CBF HAP3-TYPE SUBUNITS

| GI Accession | |
|---|---|
| Number | Brief Description |
| 3282674 | CCAAT-box binding factor HAP3 homolog [Arabidopsis thaliana] |
| 6552738 | [Arabidopsis thaliana] |
| 9758795 | Contains similarity to CCAAT-box-binding transcription |
| | factor˜gene_id: MNJ7.26 [Arabidopsis thaliana] |
| 7443520 | Transcription factor, CCAAT-binding, chain A - Arabidopsis thaliana |
| 2398529 | Transcription factor [Arabidopsis thaliana] |
| 9758792 | Contains similarity to CCAAT-box-binding transcription |
| | factor˜gene_id: MNJ7.23 [Arabidopsis thaliana] |
| 11358889 | Transcription factor NF-Y, CCAAT-binding-like protein - Arabidopis |
| | thaliana |
| 4371295 | Putative CCAAT-box-binding transcription factor [Arabidopsis thaliana] |
| 2398527 | Transcription factor [Arabidopsis thaliana] |
| 115840 | CBFA_MAIZE CCAAT-BINDING TRANSCRIPTION FACTOR SUBUNIT |
| | A (CBF-A) |
| 22380 | CAAT-box DNA binding protein subunit B (NF-YB) [Zea mays] |
| 4558662 | Putative CCAAT-box-binding transcription factor [Arabidopsis thaliana] |
| 3928076 | Putative CCAAT-box-binding transcription factor subunit [Arabidopsis |
| | thaliana] |
| 203355 | CCAAT binding transcription factor-B subunit [Rattus norvegicus] |
| 104551 | Transcription factor NF-Y, CAAT-binding, chain B - chicken |
| 2133270 | Transcription factor HAP3 - Emericella nidulans |
| 3170225 | Nuclear Y/CCAAT-box binding factor B subunit NF-YB [Xenopus laevis] |
| 115842 | CBFA_PETMA CCAAT-BINDING TRANSCRIPTION FACTOR |
| | SUBUNIT A (CBF-A) |
| 13648093 | Nuclear transcription factor Y, beta [Homo sapiens] |
| 3738293 | Putative CCAAT-box-binding transcription factor [Arabidopsis thaliana] |
| 115838 | CBFA_CHICK CCAAT-BINDING TRANSCRIPTION FACTOR SUBUNIT |
| | A (CBF-A) |
| 115840 | CBFA_MAIZE CCAAT-BINDING TRANSCRIPTION FACTOR SUBUNIT |
| | A (CBF-A) |
| 22380 | CAAT-box DNA binding protein subunit B (NF-YB) [Zea mays] |
| 4558662 | Putative CCAAT-box-binding transcription factor [Arabidopsis thaliana] |
| 3928076 | Putative CCAAT-box-binding transcription factor subunit [Arabidopsis |
| | thaliana] |
| 203355 | CCAAT binding transcription factor-B subunit [Rattus norvegicus] |
| 104551 | Transcription factor NF-Y, CAAT-binding, chain B - chicken |
| 2133270 | Transcription factor HAP3 - Emericella nidulans |
| 3170225 | Nuclear Y/CCAAT-box binding factor B subunit NF-YB [Xenopus laevis] |
| 115842 | CBFA_PETMA CCAAT-BINDING TRANSCRIPTION FACTOR |
| | SUBUNIT A (CBF-A) |
| 13648093 | Nuclear transcription factor Y, beta [Homo sapiens] |
| 3738293 | Putative CCAAT-box-binding transcription factor [Arabidopsis thaliana] |
| 115838 | CBFA_CHICK CCAAT-BINDING TRANSCRIPTION FACTOR SUBUNIT |
| | A (CBF-A) |

Other HAP3-type CBF polypeptides can be identified by homologous nucleotide and polypeptide sequence analyses. Known HAP3-type CBF subunits in one organism can be used to identify homologous subunits in another organism. For example, performing a query on a database of nucleotide or polypeptide sequences can identify homologs of a subunit of a known HAP3-type CBF complex. Homologous sequence analysis can involve BLAST or PSI-BLAST analysis of nonredundant databases using known HAP3-type CBF subunit amino acid sequences. Those proteins in the database that have greater than 40% sequence identity are candidates for further evaluation for suitability as a seed infertility factor polypeptide. If desired, manual inspection of such candidates can be carried out in order to narrow the number of candidates that may be further evaluated. Manual inspection is performed by selecting those candidates that appear to have domains suspected of being present in subunits of HAP3-type CBF complexes.

A percent identity for any subject nucleic acid or amino acid sequence relative to another “target” nucleic acid or amino acid sequence can be determined. For example, conserved regions of polypeptides can be determined by aligning sequences of the same or related polypeptides from closely related plant species. Closely related plant species preferably are from the same family. Alternatively, alignments are performed using sequences from plant species that are all monocots or are all dicots. In some embodiments, alignment of sequences from two different plant species is adequate, e.g., sequences from canola and Arabidopsis can be used to identify one or more conserved regions.

Typically, polypeptides that exhibit at least about 35% amino acid sequence identity are useful to identify conserved regions in polypeptides. Conserved regions of related proteins sometimes exhibit at least 50% amino acid sequence identity; or at least about 60%; or at least 70%, at least 80%, or at least 90% amino acid sequence identity. In some embodiments, a conserved region of target and template polypeptides exhibit at least 92, 94, 96, 98, or 99% amino acid sequence identity. Amino acid sequence identity can be deduced from amino acid or nucleotide sequence.

Highly conserved domains have been identified within HAP3-type CBF subunits. These conserved regions can be useful in identifying HAP3-type CBF subunits. The primary amino acid sequences of HAP3-type CBF subunits indicate the presence of TATA-box-binding protein association domains as well as histone fold motifs, which are important for protein dimerization. A conserved HAP 3 region derived from this sequence alignment can be represented as follows:
+EQD<2>(L,M)P(I,V)AN(V,I)<1>+IM+<2>aP<2>(A,G)K(I,V)t(D,K)(D,E) (A,S)K(E,D)<1>aQECVSErISF(I,V)(T,S)tE(A,L)<1>n+C(Q,H)<1>E(Q,K) RKT(I,V)(T,N)tnDa<2>Aa<2>LGFn<1>Y<3>L<2>ra<1>+rR, where
   +=“positive” e.g. H, K, R
   a=“Aliphatic” e.g. I,L,V,M
   t=“Tiny” e.g. T,G,A
   r=“Aromatic” e.g. F,Y,W
   n=“Negative” e.g. E,D
   p=“Polar” e.g. N,Q
   <#>=specified # of amino acids, any type
   (X,Y)=one amino acid, e.g. either X or Y

Transcription activators. A transcription activator is a polypeptide that binds to a recognition site on DNA, resulting in an increase in the level of transcription from a promoter operably linked in cis with the recognition site. Many transcription activators have discrete DNA binding and transcription activation domains. The DNA binding domain(s) and transcription activation domain(s) of transcription activators can be synthetic or can be derived from different sources (e.g., two-component system or chimeric transcription activators). In some embodiments, a two-component system transcription activator has a DNA binding domain derived from the yeast gal4 gene and a transcription activation domain derived from the VP16 gene of herpes simplex virus. In other embodiments, a two-component system transcription activator has a DNA binding domain derived from a yeast HAP1 gene and the transcription activation domain derived from VP16. Populations of transgenic organisms or cells having a first nucleic acid construct that encodes a chimeric polypeptide and a second nucleic acid construct that encodes a transcription activator polypeptide can be produced by transformation, transfection, or genetic crossing. See, e.g., WO 97/31064.

Nucleic acid expression. For expression of a sequence to be transcribed, seed infertility factor (polypeptide or nucleic acid agonist), or transcription activator, a coding sequence of the invention is operably linked to a promoter and, optionally, a recognition site for a transcription activator. As used herein, the term “operably linked” refers to positioning of a regulatory element in a nucleic acid relative to a coding sequence so as to allow or facilitate transcription of the coding sequence. For example, a recognition site for a transcription activator is positioned with respect to a promoter so that upon binding of the transcription activator to the recognition site, the level of transcription from the promoter is increased. The position of the recognition site relative to the promoter can be varied for different transcription activators, in order to achieve the desired increase in the level of transcription. Selection and positioning of promoter and transcription activator recognition site is affected by several factors, including, but not limited to, desired expression level, cell or tissue specificity, and inducibility. It is a routine matter for one of skill in the art to modulate the expression of a coding sequence by appropriately selecting and positioning promoters and recognition sites for transcription activators.

A promoter suitable for being operably linked to a transcription activator nucleic acid typically has greater expression in endosperm or embryo, and lower expression in other plant tissues. Such a promoter permits expression of the transcription during seed development, and thus, expression of a sequence to be transcribed during seed development.

A promoter suitable for being operably linked to a sequence to be transcribed can, if desired, have greater expression in one or more tissues of a developing embryo or developing endosperm. For example, such a promoter can have greater expression in the aleurone layer, parts of the endosperm such as chalazal endosperm. Expression typically occurs throughout development. If a sequence to be transcribed is targeted to endosperm and encodes a polypeptide, accumulation of the product can be facilitated by fusing certain amino acid sequences to the amino- or carboxy-terminus of the polypeptide. Such amino acid sequences include KDEL and HDEL, which facilitate targeting of the polypeptide to the endoplasmic reticulum. A histone can be fused to the polypeptide, which facilitates targeting of the polypeptide to the nucleus. Extensin can be fused to the polypeptide, which facilitates targeting to the cell wall. A seed storage protein can be fused to the polypeptide, which facilitates targeting to protein bodies in the endosperm or cotyledons.

Some suitable promoters initiate transcription only, or predominantly, in certain cell types. For example, a promoter specific to a reproductive tissue (e.g., fruit, ovule, seed, pollen, pistils, female gametophyte, egg cell, central cell, nucellus, suspensor, synergid cell, flowers, embryonic tissue, embryo, zygote, endosperm, integument, seed coat or pollen) is used. A cell type or tissue-specific promoter may drive expression of operably linked sequences in tissues other than the target tissue. Thus, as used herein a cell type or tissue-specific promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Exemplary reproductive tissue promoters include those derived from the following seed-genes: zygote and embryo LEC1; suspensor G564; maize MAC1 (see, Sheridan (1996) Genetics 142:1009-1020); maize Cat3, (see, GenBank No. L05934, Abler (1993) Plant Mol. Biol. 22:10131-1038); Arabidopsis viviparous-1, (see, Genbank No. U93215); Arabidopsis atmycl, (see, Urao (1996) Plant Mol. Biol. 32:571-57, Conceicao (1994) Plant 5:493-505); Brassica napus napin gene family, including napA, (see, GenBank No. J02798, Josefsson (1987) JBL 26:12196-1301, Sjodahl (1995) Planta 197:264-271). The ovule-specific promoters FBP7 and DEFH9 are also suitable promoters. Colombo, et al. (1997) Plant Cell 9:703-715; Rotino, et al. (1997) Nat. Biotechnol. 15:1398-1401. The nucellus-specific promoter described in Cehn and Foolad (1997) Plant Mol. Biol. 35:821-831, is also suitable. Early meiosis-specific promoters are also useful. See, Kobayshi et al., (1994) DNA Res. 1:15-26; Ji and Landgridge (1994) Mol. Gen. Genet. 243:17-23. Other meiosis-related promoters include the MMC-specific DMC1 promoter and the SYN1 promoter. See, Klimyuk and Jones (1997) Plant J. 11:1-14; Bai et al. (1999) Plant Cell 11:417-430. Other exemplary reproductive tissue-specific promoters include those derived from the pollen genes described in, for example: Guerrero (1990) Mol. Gen. Genet. 224:161-168; Wakeley (1998) Plant Mol. Biol. 37:187-192; Ficker (1998) Mol. Gen. Genet. 257:132-142; Kulikauskas (1997) Plant Mol. Biol. 34:809-814; and Treacy (1997) Plant Mol. Biol. 34:603-611). Yet other suitable reproductive tissue promoters include those derived from the following embryo genes: Brassica napus 2s storage protein (see, Dasgupta (1993) Gene 133:301-302); Arabidopsis 2s storage protein; soybean b-conglycinin; Brassica napus oleosin 20 kD gene (see, GenBank No. M63985); soybean oleosin A (see, Genbank No. U09118); soybean oleosin B (see, GenBank No. U09119); Arabidopsis oleosin (see, GenBank No. Z17657); maize oleosin 18 kD (see, GenBank No. J05212; Lee (1994) Plant Mol. Biol. 26:1981-1987; and the gene encoding low molecular weight sulfur rich protein from soybean, (see, Choi (1995) Mol. Gen, Genet. 246:266-268). Yet other exemplary reproductive tissue promoters include those derived from the following genes: ovule BEL1 (see Reiser (1995) Cell 83:735-742; Ray (1994) Proc. Natl. Acad. Sci. USA 91:5761-5765; GenBank No. U39944); central cell FIE1; flower primordia Arabidopsis APETALA1 (AP1) (see, Gustafson-Brown (1994) Cell 76:131-143; Mandrel (1992) Nature 360:273-277); flower Arabidopsis AP2 (see, Drews (1991) Cell 65:991-1002; Bowman (1991) Plant Cell 3:749-758); Arabidopsis flower ufo, expressed at the junction between sepal and petal primordia (see, Bossinger (1996) Development 122:1093-1102); fruit-specific tomato E8; a tomato gene expressed during fruit ripening, senescence and abscission of leaves and flowers (Blume (1997) Plant J. 12:731-746); and pistil-specific potato SK2 (Ficker (1997) Plant Mol. Biol. 35:425-431). See also, WO 98/08961; WO 98/28431; WO 98/36090; U.S. Pat. No. 5,907,082; U.S. Pat. Nos. 6,320,102; 6,235,975; and WO 00/24914. Suitable promoters also include those that are inducible, e.g., by tetracycline (Gatz, 1997), steroids (Aoyama and Chua, 1997), and ethanol (Slater et al. 1998, Caddick et al., 1998).

Nucleic acids. A nucleic acid for use in the invention may be obtained by, for example, DNA synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids for use in the invention may be detected by techniques such as ethidium bromide staining of agarose gels, Southern or Northern blot hybridization, PCR or in situ hybridizations. Hybridization typically involves Southern or Northern blotting. See e.g., Sambrook et al., 1989, Molecular Cloning: A Laboratory Manual, 2^{nd} Edition, Cold Spring Harbor Press, Plainview, N.Y., sections 9.37-9.52. Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Methods for Making a Polypeptide

In another aspect, the invention features a method for making a polypeptide. The method involves obtaining seed produced as described above. Such seed are infertile and can be identified by, e.g., the presence of at least the three nucleic acids described above. In some embodiments, there are two transcription activators present in the male-fertile plants and, therefore, four nucleic acids, as described above. A practitioner can obtain seed of the invention by harvesting seeds from both the male-sterile and male-fertile plants, or harvesting seeds solely from the male-sterile plants. The choice depends upon, inter alia, whether the two types of parent plants are planted in rows or are randomly interplanted. However, either type of harvesting is encompassed by the invention. In some embodiments, seeds are obtained by purchasing them from a grower. In some embodiments, a practitioner permits the male-fertile plants to pollinate the male-sterile plants prior to harvesting.

The method also involves extracting the preselected polypeptide, or an endogenous polypeptide, from the seed. Typically, such seeds have a statistically significant increase in the amount of the preselected polypeptide relative to seeds that do not contain or express the first nucleic acid. The choice of techniques to be used for carrying out extraction of a preselected polypeptide will depend on the nature of the polypeptide. For example, if the preselected polypeptide is an antibody, non-denaturing purification techniques may be used. On the other hand, if the preselected polypeptide is a high methionine zein, denaturing techniques may be used. The degree of purification can be adjusted as desired, depending on the nature of the preselected or endogenous polypeptide. For example, an animal feed having an increased amount of an endogenous polypeptide may have no purification, whereas a preselected antibody polypeptide may have extensive purification.

Plants and Seeds

Plants Techniques for introducing exogenous nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures by techniques known to those skilled in the art. Transgenic plants can be entered into a breeding program, e.g., to introduce a nucleic acid into other lines, to transfer a nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, and subsequent generation plants, or seeds formed on BC₁, BC₂, BC₃, and subsequent generation plants. Seeds produced by a transgenic plant can be grown and then selfed (or outcrossed and selfed) to obtain seeds homozygous for the nucleic acid encoding a novel polypeptide.

A suitable group of plants with which to practice the invention include dicots, such as safflower, alfalfa, soybean, rapeseed (high erucic acid and canola), or sunflower. Also suitable are monocots such as corn, wheat, rye, barley, oat, rice, millet, amaranth or sorghum. Also suitable are vegetable crops or root crops such as broccoli, peas, sweet corn, popcorn, tomato, beans (including kidney beans, lima beans, dry beans, green beans) and the like. Also suitable are fruit crops such as peach, pear, apple, cherry, orange, lemon, grapefruit, plum, mango and palm. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panicum, Pannesetum, Persea, Phaseolus, Pinus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna and Zea.

Plants of the first type are male-sterile, e.g., pollen is either not formed or is nonviable. Suitable male-sterility systems are known, including cytoplasmic male sterility (CMS), nuclear male sterility, genetic male sterility, and molecular male sterility wherein a transgene inhibits microsporogenesis and/or pollen formation. Female parent plants containing CMS are particularly useful. In the case of Brassica species, CMS can be, for example of the ogu, nap, pol, mur, or tour type. See, e.g., U.S. Pat. Nos. 6,399,856, 6,262,341; 6,262,334; 6,392,119 and 6,255,564. In the case of corn, a number of different methods of conferring male sterility are available, such as multiple mutant genes at separate locations within the genome that confer male sterility. In addition, one can use transgenes to silence one or more nucleic acid sequences necessary for male fertility. See, U.S. Pat. Nos. 4,654,465, 4,727,219, and 5,432,068. See also, EPO publication no. 329, 308 and PCT application WO 90/08828.

One can also use gametocides. Gametocides are chemicals that affect cells critical to male fertility. Typically, a gametocide affects fertility only in the plants to which the gametocide is applied. Application of the gametocide, timing of the application and genotype can affect the usefulness of the approach. See, U.S. Pat. No. 4,936,904.

Articles of Manufacture

A plant seed composition of the invention contains seeds of the first type of plant and of the second type of plant. Seeds of the first type of plant typically are of a single variety, as are seeds of the second type of plant.

The proportion of seeds of each type of plant in a composition is measured as the number of seeds of a particular type divided by the total number of seeds in the composition, and can be formulated as desired to meet requirements based on geographic location, pollen quantity, pollen dispersal range, plant maturity, choice of herbicide, and the like. The proportion of the first variety can be from about 70 percent to about 99.9 percent, e.g., 90%, 91%, 92%, 93%, 94%, 95%, 96%, 97%, 98%, or 99%. The proportion of the second type can be from about 0.1 percent to about 30 percent, e.g., 0.5%, 1%, 2%, 5%, 10%, 15%, or 30%. When large quantities of a seed composition are formulated, or when the same composition is formulated repeatedly, there may be some variation in the proportion of each type observed in a sample of the composition, due to sampling error. Sampling error is known from statistics. In the present invention, such sampling error typically is about ±5% of the expected proportion, e.g., 90%±4.5%, or 5%±0.25%.

For example, a seed composition of the invention can be made from two corn varieties. A first corn variety can constitute 92% of the seeds in the composition and be male-sterile, and carry a first nucleic acid encoding one or more polypeptides involved in the synthesis of poly(3-hydroxybutyrate-co-3-hydroxyvalerate. A second corn variety can constitute 8% of the seed in the composition and be male-fertile, and carry a third nucleic acid encoding a transcription activator that recognizes a transcription recognition site operably linked to a nucleic acid encoding a preselected polypeptide. Thus, such a seed composition can be used to grow plants that are suitable for practicing a method of the invention.

Typically, a substantially uniform mixture of seeds of each of the types is conditioned and bagged in packaging material by means known in the art to form an article of manufacture. Such a bag of seed preferably has a package label accompanying the bag, e.g., a tag or label secured to the packaging material, a label printed on the packaging material or a label inserted within the bag. The package label indicates that the seeds therein are a mixture of varieties, e.g., two different varieties. The package label may indicate that plants grown from such seeds are suitable for making an indicated preselected polypeptide. The package label also may indicate the seed mixture contained therein incorporate transgenes that provide biological containment of the transgene encoding the preselected polypeptide.

Plants grown from the varieties in a seed composition of the invention typically have the same or very similar maturity, i.e., the same or very similar number of days from germination to crop seed maturation. In some embodiments, however, one or more varieties in a seed composition of the invention can have a different relative maturity compared to other varieties in the composition. For example, the first type of plants grown from a seed composition can be classified as having a 105 day relative maturity, while the second type of plants grown from the seed composition can be classified as having a 110 day relative maturity. The presence of plants of different relative maturities in a seed composition can be useful as desired to properly coordinate optimum pollen receptivity of the first type of plants with optimum pollen shed from the second type of plants. Relative maturity of a variety of a given crop species is classified by techniques known in the art.

The invention is further described in the following examples, which do not limit the scope of the invention described in the claims.

### EXAMPLES

### Example 1

### Chimeric LEC2 Nucleic Acid Construct

A chimeric LEC2 gene construct, designated pLEC2, was made using standard molecular biology techniques. The construct contains the coding sequence for the Arabidopsis LEC2 polypeptide. pLEC2 contains 5 binding sites for the DNA binding domain upstream activation sequence of the Hap1 transcription factor (UAS_{Hap1}) located 5′ to and operably linked to a CaMV35S minimal promoter. The CaMV35S minimal promoter is located 5′ to and operably linked to the LEC2 coding sequence. The construct contains an OCS polyA transcription terminator sequence operably linked to the 3′ end of the LEC2 coding sequence. The binding of a transcription factor that possesses a Hap1 DNA binding domain to the UAS_{Hap1} is necessary for transcriptional activation of the LEC2 chimeric gene.

### Example 2

### Transgenic Rice Plants

The pLEC2 plasmid was introduced into the Japonica rice cultivar Kitaake by Agrobacterium tumefaciens mediated transformation using techniques similar to those described in U.S. Pat. No. 6,329,571. Transformants were selected based on resistance to the herbicide bialophos, conferred by a bar gene present on the introduced nucleic acid. After selfing to homozygosity for 3 generations, several transformed plants, designated pLEC2-3-11-10, pLEC2-3-11-12, pLEC2-3-11-13, pLEC2-3-12-2, pLEC2-3-12-4, were selected for further study.

A construct designated pCR19, containing a chimeric Hap1-VP16 gene and a green fluorescent protein (GFP) reporter gene was introduced into the Kitaake cultivar by the same technique. The chimeric Hap1-VP16 gene contained a rice ubiquitin minimal promoter operably linked to the 5′ end of the Hap1-VP16 coding sequence and an NOS polyA terminator operably linked to the 3′ end of the Hap1-VP16 coding sequence. The amino acid sequence of the HAP1 portion of the Hap1-VP16 transcription activator is that of the yeast Hap1 gene. The GFP reporter gene included 5 copies of a UAS_{HAP1} upstream activator sequence element operably linked 5′ to the GFP coding sequence and an OCS polyA terminator operably linked 3′ to the GFP coding sequence. Transformants were selected based on bialophos resistance conferred by a bar gene, and then screened for plants in which expression of GFP was targeted to the embryo. After selfing for 2 generations and verifying embryo-specific expression of the Hap1-VP16 coding sequence, 2 heterozygous transformed plants, designated CR19-60-1 and CR19-60-2, were selected for further study. By microscopic evaluation, these plants showed high levels of GFP expression in developing embryos, little or no GFP expression in endosperm, and low levels of GFP expression in seedlings.

Rice plants homozygous for the LEC2 transgene were crossed as females with CR19-60-1 and CR19-60-2 plants. Samples of the developing F₁ embryos were collected at 5 days, 8 days, and 12 days after pollination.

Nine embryos collected at 5 days after pollination were observed under a dissecting microscope and a fluorescent microscope. The presence or absence of the Hap1-VP16 chimeric gene was determined based on the presence or absence of GFP reporter gene activity as visualized with a UV-equipped microscope. Four embryos were found to have received the Hap1-VP16 gene. The development of these embryos was delayed and was equivalent to the development of a corresponding control embryo at 3 days after pollination. In addition, the scutellum and first leaf were found to be fused. The other 5 embryos did not have the Hap1-VP16 chimeric gene and showed normal development.

At 8 days after pollination, developing embryos were placed on phytohormone-free MS germination media and germination was observed for up to 24 days. Of 10 embryos evaluated, 1 embryo contained both Hap1-VP16 and LEC2. This embryo was found to have lost the ability to germinate. The other 9 control embryos did not contain the Hap1-VP16 chimeric gene, and formed normal seedlings.

Seventeen embryos collected at 12 days after pollination were dissected by cutting longitudinally through the embryonic axis. Dissected embryos were then observed under a dissecting microscope, and it was found that the 7 Hap1-VP16 expressing embryos formed multiple shoots but no root primordium initiation. In addition, the leaves were not well developed. The other 10 embryos did not contain Hap1-VP16 and showed normal shoot, root and leaf differentiation.

Mature F₁ seed was collected 27 days after pollination and allowed to dry. Thirteen seeds contained both pLEC2 and the activation construct CR19. Twenty five seeds contained the pLEC2 construct only. F₁ seeds, together with control seeds, were germinated on agar plates containing hormone-free 0.5× Murashige and Skoog (MS) salts, 1.5 percent sucrose and 0.25 percent Gelrite. Germination efficiency was scored 19 days later. Seeds containing Hap1-VP16 and expressing LEC2 were completely infertile and had 0% germination, whereas control seeds had 100% germination. These data indicate that embryo-targeted LEC2 expression results in infertile seed.

A similar experiment was conducted using Hap1-VP16 lines selected for targeting to the endosperm. Two different endosperm-specific promoters were used to drive Hap1-VP16. Transgenic plants obtained from each transformation expressed GFP targeted to endosperm only. Plants homozygous for Hap1-VP16 and GFP were obtained after selfing for 2 generations and used to pollinate the pLEC2 homozygous plants. Mature F₁ seed was collected and allowed to dry. F1 Seeds containing Hap1-VP16 and expressing LEC2 were fertile and had a normal germination rate on the phytohormone-free MS medium. These data indicate that endosperm-targeted LEC2 expression results in fertile seed.

### Example 3

### Transgenic Soybean Plants

A soybean plant homozygous for a transgene comprising the LEC2 coding sequence operably linked to 5 copies of a UAS_{Hap1} and a 35S minimal promoter was crossed as a female, using pollen from a soybean plant homozygous for a transgene comprising a HAP1-VP16 polypeptide operably linked to an embryo-targeted regulatory sequence. The soybean plant used as a female also is homozygous for a transgene comprising the coding sequence for a tumor necrosis factor receptor polypeptide, operably linked to 5 copies of a UAS_{Hap1} and a 35S minimal promoter. See, e.g., U.S. Pat. No. 6,541,610.

At maturity, F₁ seeds are collected and stored under standard conditions. Any tumor necrosis factor receptor expressed in the F₁ seeds is extracted. At 7, 14, and 21 days after pollination, some of the embryos and seeds developing on F₁ plants are examined under a microscope. Mature seed also are scored for viability and germination and tested for the presence of tumor necrosis factor receptor coding sequence by PCR. The procedure is repeated using corn plants instead of soybean plants.

It is to be understood that while the invention has been described in conjunction with the detailed description thereof, the foregoing description is intended to illustrate and not limit the scope of the invention.

1. A method for making infertile seed, said method comprising:
a) permitting seed development to occur on a plurality of first plants that have been pollinated by a plurality of second plants, wherein said first plants are male-sterile and comprise first and second nucleic acids, said first nucleic acid comprising a first transcription activator recognition site and a first promoter, said first recognition site and said first promoter operably linked to a sequence to be transcribed, said second nucleic acid comprising a second transcription activator recognition site and a second promoter, said second recognition site and said second promoter operably linked to a coding sequence that results in seed infertility,
wherein said second plants are male-fertile and comprise at least one activator nucleic acid comprising at least one coding sequence for a transcription activator that binds to at least one of said recognition sites, each said at least one transcription activator coding sequence having a promoter operably linked thereto, and wherein said seeds are infertile.

2. The method of claim 1, wherein said at least one activator nucleic acid is a single nucleic acid encoding a single transcription activator that binds said first and said second recognition sites.

3. The method of claim 2, wherein said promoter for said transcription activator is seed-specific.

4. The method of claim 3, wherein said promoter for said transcription activator is an Arabidopsis LEC1 promoter.

5. The method of claim 2, wherein said promoter for said transcription activator is chemically inducible.

6. The method of claim 1, wherein said at least one activator nucleic acid is a single nucleic acid encoding a first transcription activator that binds said first recognition site and encoding a second transcription activator that binds said second recognition site.

7. The method of claim 6, wherein said promoter for said first transcription activator is a constitutive promoter and said promoter for said second transcription activator is a seed-specific promoter.

8. The method of claim 7, wherein said promoter for said first transcription activator is a maize ubiquitin promoter.

9. The method of claim 1, wherein said plants are dicotyledonous plants.

10. The method of claim 1, wherein said plants are monocotyledonous plants.

11. The method of claim 1, further comprising the step of harvesting said seeds.

12. The method of claim 1, wherein said plurality of first plants is cytoplasmically male-sterile.

13. The method of claim 1, wherein said plurality of first plants is male-sterile due to nuclear male sterility.

14. The method of claim 1, wherein said sequence to be transcribed encodes a preselected polypeptide.

15. The method of claim 14, wherein said seeds have a statistically significant increase in the amount of said preselected polypeptide relative to seeds that do not contain or express said first nucleic acid.

16. The method of claim 15, wherein said preselected polypeptide is an antibody.

17. The method of claim 15, wherein said preselected polypeptide is an enzyme.

18. The method of claim 1, wherein said sequence causing seed infertility encodes a seed infertility polypeptide.

19. The method of claim 18, wherein said seed infertility polypeptide is a loss-of-function mutant FIE polypeptide.

20. The method of claim 18, wherein said seed infertility polypeptide is an ANT polypeptide.

21. The method of claim 18, wherein said seed infertility polypeptide is a LEC1 polypeptide.

22. A method for making a polypeptide, said method comprising:
a) obtaining seed produced by pollination of a male-sterile plant, said seed comprising: i) a first nucleic acid comprising a first recognition site for a transcription activator and a first promoter, said first recognition site and said first promoter operably linked to a sequence to be transcribed; ii) a second nucleic acid comprising a second recognition site for a transcription activator and a second promoter, said second recognition site and said second promoter operably linked to a sequence causing seed infertility; and iii) at least one activator nucleic acid comprising at least one coding sequence for a transcription activator that binds to at least one of said recognition sites, each said at least one transcription activator having a promoter operably linked thereto, wherein said seeds are infertile and have a statistically significant increase in the amount of an endogenous polypeptide relative to seeds that do not contain or express said first nucleic acid.

23. The method of claim 22, wherein each said promoter for said one or more activator nucleic acids is an Arabidopsis LEC1 promoter.

24. The method of claim 22, wherein said plurality of first plants and said plurality of second plants are randomly interplanted.

25. The method of claim 22, wherein said sequence causing seed infertility encodes a seed infertility polypeptide.

26. The method of claim 22, further comprising the step of extracting said preselected polypeptide from said seeds.

27. A method for making a polypeptide, said method comprising:
a) permitting a plurality of first, male-sterile, plants to be pollinated by a plurality of second plants, each of said first plants comprising: i) a first nucleic acid comprising a first transcription activator recognition site and a first promoter, said first recognition site and said first promoter operably linked to a nucleic acid encoding a preselected polypeptide; and ii) a second nucleic acid comprising a second transcription activator recognition site and a second promoter, said second recognition site and said second promoter operably linked to a sequence causing seed infertility, each of said second plants comprising at least one activator nucleic acid encoding at least one transcription activator that binds to at least one of said recognition sites, each said at least one transcription activator nucleic acid having a promoter operably linked thereto; and
b) harvesting seeds from said plurality of first plants, wherein said seeds are infertile and have a statistically significant increase in said preselected polypeptide relative to seeds that do not contain or express said first nucleic acid.

28. An article of manufacture comprising:
a) a container;
b) a first type of seeds within said container, said first type of seeds comprising at least one first nucleic acid comprising: i) a first transcription activator recognition site and a first promoter, said first recognition site and said first promoter operably linked to a sequence to be transcribed; and ii) a second transcription activator recognition site and a second promoter, said second recognition site and said second promoter operably linked to a sequence causing seed infertility, wherein plants grown from said first type of seeds are male-sterile; and
c) a second type of seeds within said container, said second type of seeds comprising at least one activator nucleic acid encoding at least one transcription activator that binds to at least one of said recognition sites, each said at least one transcription activator having a promoter operably linked thereto, wherein plants grown from said second type of seeds are male-fertile.

29. The article of claim 28, wherein said sequence to be transcribed is a preselected polypeptide.

30. The article of claim 28, wherein the ratio of said first type of seeds to said second type of seeds is about 70:30 or greater.

31. The article of claim 28, wherein said at least one first nucleic acid comprises a nucleic acid comprising said first transcription activator recognition site, said first promoter and said sequence to be transcribed, and a different nucleic acid comprising said second transcription activator recognition site, said second promoter and a seed infertility polypeptide coding sequence.

32. The article of claim 28, wherein said at least one activator nucleic acid encodes a transcription activator that binds to said first recognition site, and a different transcription activator that binds to said second recognition site.

33. The article of claim 32, wherein said promoter for said transcription activator that binds said first recognition site is a seed-specific promoter and said promoter for said transcription activator that binds to said second recognition site is a maize ubiquitin promoter.

34. The article of claim 28, wherein said first and said second types of seeds are dicotyledonous seeds.

35. The article of claim 28, wherein said first and said second types of seeds are monocotyledonous seeds.

36. The article of claim 28, wherein said first type of seeds are cytoplasmically male sterile.

37. A nucleic acid construct comprising:
a) a first transcription activator recognition site and a first promoter, said first recognition site and said first promoter operably linked to a sequence to be transcribed; and
b) a second transcription activator recognition site and a second promoter, said second recognition site and said second promoter operably linked to a sequence causing seed infertility.

38. The nucleic acid construct of claim 37, wherein said sequence causing seed infertility is transcribed into a FIE antagonist.

39. The nucleic acid construct of claim 37, wherein said FIE antagonist is an antisense RNA.

40. The nucleic acid construct of claim 37, wherein said FIE antagonist is a ribozyme

41. The nucleic acid construct of claim 37, wherein said FIE antagonist is a chimeric polypeptide comprising a polypeptide segment exhibiting histone acetyltransferase activity fused to a polypeptide segment exhibiting activity of a subunit of a chromatin-associated protein complex having histone deacetylase activity.

42. The nucleic acid construct of claim 37, wherein said sequence to be transcribed encodes a preselected polypeptide.

43. The nucleic acid construct of claim 42, wherein said preselected polypeptide is an antibody.

44. The nucleic acid construct of claim 42, wherein said preselected polypeptide has immunogenic activity in a mammal.

45. The nucleic acid construct of claim 42, wherein said preselected polypeptide is an enzyme.

46. The nucleic acid construct of claim 45, wherein said preselected polypeptide is glucose-6-phosphate dehydrogenase.

47. The nucleic acid construct of claim 45, wherein said preselected polypeptide is alpha-amylase.

48. The nucleic acid construct of claim 37, wherein said sequence causing seed infertility encodes ANT.

49. The nucleic acid construct of claim 37, wherein said sequence causing seed infertility encodes LEC1.

50. A plant comprising:
a) a first nucleic acid comprising a first transcription activator recognition site and a first promoter, said first recognition site and said first promoter operably linked to a sequence to be transcribed,
b) a second nucleic acid comprising a second transcription activator recognition site and a second promoter, said second recognition site and said second promoter operably linked to a sequence causing seed infertility.

51. The plant of claim 50, wherein said plant is male-sterile.

52. The plant of claim 50, wherein said plant is cytoplasmically male sterile.

53. The plant of claim 50, wherein said plant is male sterile due to nuclear male sterility.

54. The plant of claim 50, wherein said plant is genetically male sterile.

55. The plant of claim 50, wherein said first and second nucleic acids are a single nucleic acid molecule.

56. The plant of claim 50, wherein said plant is a dicotyledonous plant.

57. The plant of claim 50, wherein said plant is a monocotyledonous plant.

58. The plant of claim 50, wherein said sequence to be transcribed encodes a preselected polypeptide.
